(19) United States
(12) Patent Application Publication
(10) Pub. No.: US 2019/0232059 A1
(43) Pub. Date: Aug. 1, 2019

(54) Targeting Alpha Oscillations with Transcranial Alternating Current Stimulation (tACS) for the Treatment of Major Depressive Disorder (MDD)

(71) Applicant: The University of North Carolina at Chapel Hill, Chapel Hill, NC (US)

(72) Inventor: Flavio Frohlich, Chapel Hill, NC (US)

(21) Appl. No.: 16/257,444

(22) Filed: Jan. 25, 2019

Related U.S. Application Data
(60) Provisional application No. 62/622,384, filed on Jan. 26, 2018.

(51) Int. Cl. A61N 1/36; A61N 1/04; A61B 5/048

(52) U.S. Cl. CPC A61N 1/36025; A61B 5/048; A61N 1/0476; A61N 1/0456

(57) ABSTRACT

In one aspect, methods of treating major depressive disorder (MDD) employing transcranial alternating current stimulation are described herein. A method of treating MDD in a patient comprises reducing alpha oscillations in one or more frontal brain regions of the patient via administering transcranial alternating current stimulation (tACS) to the patient at a frequency within the alpha frequency band.

### RELATED APPLICATION DATA

The present invention claims priority pursuant to 35 U.S.C. § 119(e) to U.S. Provisional Patent Application Ser. No. 62/622,384 filed Jan. 26, 2018 which is incorporated herein by reference in its entirety.

### STATEMENT OF GOVERNMENT RIGHTS

This invention was made with government support under Grant Number MH105574 awarded by the National Institutes of Health. The government has certain rights in the invention.

### FIELD

The present invention relates to methods of treating major depressive disorder (MDD) and, in particular, to treating MDD via administration of transcranial alternating current stimulation (tACS) to patients.

### BACKGROUND

Major Depressive Disorder (MDD) is a common, severe psychiatric illness that has a lifetime prevalence of about 16.6% in adults and results in the highest burden of disability among all mental and behavioral disorders. Current recommended drug therapies are associated with suboptimal remission rates and, oftentimes, undesirable side effects. Furthermore, the effects of drugs on the brain are widespread, and interventions that can target specific deficiencies in brain activity may permit greater therapeutic precision.

### SUMMARY

In one aspect, methods of treating major depressive disorder (MDD) employing transcranial alternating current stimulation are described herein. Briefly, a method of treating MDD in a patient comprises reducing alpha oscillations in one or more frontal brain regions of the patient via administering transcranial alternating current stimulation (tACS) to the patient at a frequency within the alpha frequency band.

As described further herein, frontal brain regions can be targeted by the tACS. Left and right frontal brain regions, for example, can be individually targeted or simultaneously targeted by the tACS. In some embodiments, tACS is synchronously administered to left and right frontal brain regions. tACS can be administered at any frequency in the alpha band (8-12 Hz). In some embodiments, tACS is administered at 10 Hz. Alternatively, the peak alpha oscillation frequency of the patient can be measured or determined prior to administration of the tACS. The tACS is then administered to the patient at this peak oscillation frequency or substantially near this peak oscillation frequency. In being substantially near, the frequency of the tACS can deviate up to 20 percent or up to 10 percent from the peak alpha oscillation frequency of the patient. In this way, administration of the tACS can be tailored to the individual patient. An individual's alpha frequency can be determined through a variety of processes, including collection of two minutes of eyes-closed resting-state EEG data before each session of tACS administration.

tACS is generally administered to the patient via electrodes placed on the patient's, scalp. Any arrangement and/or number of electrodes consistent with reducing alpha oscillations in frontal brain region(s) can be employed in the administration of tACS. In some embodiments, for example, a three-electrode arrangement is used. A three-electrode arrangement can include two anodes and one cathode. The two anodes can be placed over various frontal brain regions. In some embodiments, one anode is positioned at F3 and the other anode is positioned at F4. The cathode can also have any desired placement. The cathode, for example, can be placed over the central lobe, such as along the mid-line (Cz). FIG. 1A illustrates electrode placement for tACS administration in some embodiments.

tACS stimulation is generally administered in a sine waveform of desired amplitude. In some embodiments, amplitude and phase of the stimulation can vary between the electrodes. The anodes, for example, can be in-phase while the cathode is at opposite phase at any point in the stimulation. Moreover, the sine waveform of the cathode can have a larger or smaller amplitude than the sine waveform of the anodes. Alternatively, the sine waveforms of the electrodes have equal amplitudes.

tACS stimulation within the alpha frequency band can be administered for any desired time period and at any desired interval consistent with reducing alpha oscillations in one or more frontal brain regions. In some embodiments, tACS is administered for a time period of 30 minutes to 90 minutes. tACS can be administered daily or over any interval of days for an initial treatment period to reduce alpha oscillations in frontal brain region(s). As detailed in the examples herein, tACS can be administered daily for an initial treatment period of at least 5 days, in some embodiments. The initial treatment period can vary according to the individual patient and is generally concluded when the patient exhibits sufficient reduction of alpha oscillations in one or more frontal brain regions. Sufficient reduction of alpha oscillations can be determined by high-density electroencephalograms (hdEEG). A reduction in alpha oscillation power in a frontal brain region of 0.5-4 dB, for example, can constitute sufficient reduction in some embodiments. Alternatively, sufficient reduction in alpha oscillations can be determined by improvement in MDD symptoms experienced by the patient. In some embodiments, at least a 50 percent reduction in MDD symptoms from a baseline measurement can constitute sufficient reduction in alpha oscillations. The baseline measurement can be determined prior to commencement of the initial treatment period. Baseline measurement can also be reestablished if the patient has not received tACS stimulation for a specified period of time. Symptom improvement can be assessed by using the Montgomery-Åsberg Depression Rating Scale (MADRS).

Methods of treating MDD described herein, in some embodiments, can comprise increasing alpha oscillations in one more right hemispherical regions of the brain. Increasing alpha oscillations in one or more right hemispherical regions can assist in reducing alpha oscillation disparities or imbalance between left and right hemispheres of the patient's brain. In some embodiments, alpha oscillations are increased in one or more right frontal regions of the brain. Administration of tACS at a frequency in the alpha band can increase alpha oscillations in right hemispherical region(s), in some embodiments. Specific right hemispherical regions of the brain can be individually or selectively targeted by the tACS. In other embodiments, right hemispherical brain regions are targeted by the tACS in conjunction with left hemispherical region(s). Increases in alpha oscillations in one or more right hemispherical regions can be independent of any reductions in alpha oscillations in one or more left hemispherical regions. Alternatively, increases in alpha oscillations in one or more right hemispherical regions of the brain can occur in conjunction with reductions in alpha oscillations in one or more left hemispherical regions.

In some embodiments, a method of treating MDD described herein further comprises administering tACS to the patient at one or more intervals after the initial treatment period. Administration of tACS after the initial treatment period can be considered a maintenance period. Maintenance periods can last for any desired time period including, weeks, months or years. Administration of tACS within the maintenance period can occur at any desired interval. Specific parameters of a maintenance period are largely determined according to the particular needs of an individual patient. In some embodiments, administration of tACS in the maintenance period can maintain the reduction in alpha oscillations achieved during the initial treatment period.

As described herein, administration of tACS in the alpha frequency band reduces alpha oscillations in or more frontal brain regions. In some embodiments, reducing the alpha oscillations reduces alpha oscillation imbalance in left dorsolateral prefrontal cortex. Additionally, alpha oscillations in the left and right frontal brain regions can be substantially balanced after the reduction of frontal region alpha oscillations. In being substantially balanced, alpha oscillation power between left and right frontal brain regions differs by less than 10 percent or less than 5 percent.

These and other embodiments are further described in the following detailed description.

### BRIEF DESCRIPTION OF THE DRAWINGS

FIG. 1(A)—Stimulation configuration for all participants. Two stimulators were used, one connected to the electrode over F3, one connected to the electrode over F4, and both connected to the electrode over Cz. The red electrodes (F3 and F4) are the anode and the blue return electrode over Cz is the cathode.

FIG. 1(B)—Sham and active stimulation paradigms. Ramp-in and ramp-out is 20 seconds for all conditions, with 40 seconds of active stimulation for Sham stimulation, 2400 seconds of active stimulation for 10 Hz-tACS and 40 Hz-tACS. Anodes (F3 and F4) and cathode (Cz) are at opposite phase at any given point during stimulation.

FIG. 1(C) Electric field simulation: 2D (top) and 3D (bottom) representation (HD-Explore, Soterix Medical, New York, N.Y., USA).

FIG. 2—Individualized scores per participant for the MADRS, HDRS, and BDI. Scores were normalized based on a ratio in comparison to baseline scores. Averages include standard error bars. Dashed line in each figure represents the threshold for response (i.e., at least a 50% reduction in symptoms from baseline). Note that in the 9 graphs on the right, each line represents an individual participant.)

FIG. 3(A)—Changes in EEG Alpha Power in Eyes Open Condition—Mean alpha power change at topographical region level at Day 5. Error bars denote standard error of mean. Black filled circles denote electrodes that showed significant change relative to Day 1.

FIG. 3(B)—Changes in EEG Alpha Power in Eyes Open Condition—Mean alpha power change at topographical region level at F2. Error bars denote standard error of mean.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention.

Numerous modifications and adaptations will be readily apparent to those of skill in the art without departing from the spirit and scope of the invention.

A pilot double-blind study to evaluate the feasibility, safety, and efficacy of tACS as a treatment for the symptoms of depression was conducted. Patients diagnosed with MDD were randomized to one of three arms to compare 10 Hz-tACS, 40 Hz-tACS, and active sham stimulation. The investigation of a second, different stimulation frequency (i.e., 40 Hz) served to assess if symptom and electrophysiological changes were frequency-dependent or merely stimulation-dependent. The tACS intervention comprised 40 minutes of daily stimulation for five consecutive days. The primary outcome was the change in Montgomery-Åsberg Depression Rating Scale (MADRS) score from baseline to the final follow-up study visit four weeks after completion of the intervention. To understand how tACS affects brain activity, alpha power changes were measured using high density electroencephalography (hdEEG) as our secondary outcome.

### Methods and Materials

This study was a double-blind, randomized, sham-controlled pilot clinical trial conducted at The University of North Carolina at Chapel Hill from May 2015 to June 2017 and registered at ClinicalTrials.gov (NCT02339285). The study was approved by the Biomedical Institutional Review Board at UNC Chapel Hill (IRB #14-1622) and used a Data Safety Monitoring Board (DSMB) through the North Carolina Translational & Clinical Studies Institute to ensure participant safety. Bi-annual reviews of blinded data and adverse events were submitted to the DSMB. All participants provided written informed consent prior to all study-related activities.

### Participants

A total of 32 people (27 female; aged 36.69±13.08 years) diagnosed with unipolar, non-psychotic major depressive disorder (confirmed with the M.I.N.I. International Neuropsychiatric Interview), with a Hamilton Depression Rating Scale (HDRS) of >8 and low suicide risk, defined as scoring <3 on the Suicide Item on the HDRS, were randomized in this trial. Previous treatments include medication (94% reported) and therapy (69% reported), indicating the enrolled participants in this sample have attempted to treat their depression prior to enrollment. Of the 32 enrolled participants (defined as intent-to-treat, or ITT, sample), 26 completed all study visits as designed (defined as per-protocol, or PP, sample; see CONSORT). Screened participants were excluded from participation for the following reasons: concurrent anticonvulsant medications or daily treatments with benzodiazepines (limited as-needed use that was discontinued more than 48 hours prior to a study session was allowed); DSM-IV diagnosis of alcohol or substance dependence (other than nicotine) within the last 12 months; current Axis I mood or psychotic disorder other than MDD; lifetime comorbid psychiatric bipolar or psychotic disorder; eating disorder (current or within the past 6 months); obsessive-compulsive disorder (lifetime); post-traumatic stress disorder (current or within the last 6 months); attention-deficit/hyperactivity disorder (currently under treatment); history of significant head injury or traumatic brain injury, prior brain surgery, or any brain devices/implants; history of seizures, unstable medical illness, or pregnancy. Although not an exclusion criterion, none of the participants were left-handed (Edinburgh Handedness Inventory, 82.8±24.9). Screened participants were not excluded for use of antidepressants, and 38% of participants were on at least one antidepressant at the time of enrollment. To control for changes in medication, participants were required to be at least 6 weeks stable on their antidepressants.

### Study Schedule

Participants who completed the study attended a total of 8 sessions. Inclusion and exclusion criteria were assessed with a preliminary phone screening and then more extensively at the initial session with the study coordinator. At the initial session, participants signed consent and completed several questionnaires (demographics, Edinburgh handedness Fquestionnaire, ‘Hunter Beliefs About Treatment Questionnaire,’ used with permission of the UCLA Laboratory of Brain, Behavior, and Pharmacology, ©2005, 2017 UC Regents). In addition, the study coordinator administered the M.I.N.I. and the HDRS to confirm eligibility. Prior to randomization, eligible participants also met with an experienced mood disorders clinician (D.R.) to further assess their clinical symptoms and to verify the participants met the inclusion criteria. Once eligibility was confirmed, participants returned for 5 consecutive days of treatment (Day 1 to Day 5). Baseline scores for all assessments were completed on Day 1. Participants also attended a 2 week follow-up and a 4 week follow-up after they completed the week of stimulation.

### Randomization

Participants were randomized into three study arms (10 Hz-tACS, n=10; 40 Hz-tACS, n=11; and active sham at 10 Hz, n=11). Intervention type was based on study codes prepared by a member of the research lab who was not otherwise associated with the study and codes were randomized such that no more than 3 participants in a row received the same intervention. All authors and members of the research team were unaware of the group assignments until completion of the entire study. To administer stimulation in a double-blind fashion, we developed a custom Matlab-controlled computer interface (Mathworks, Natick, Mass.; NIDaq USB 6001; National Instruments, TX, USA) to control two Neuroconn DC plus stimulators (Neuroconn Ltd., Ilmenau, Germany) that delivered the stimulation based on the study code entered. To ensure that the correct waveform was applied for each session, this interface recorded the applied waveform for subsequent verification by a group member not associated with the study.

### Stimulation

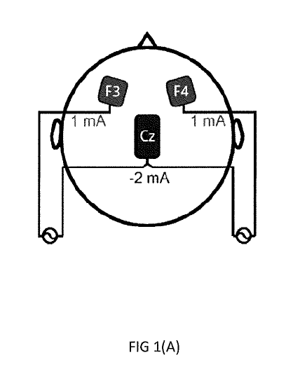

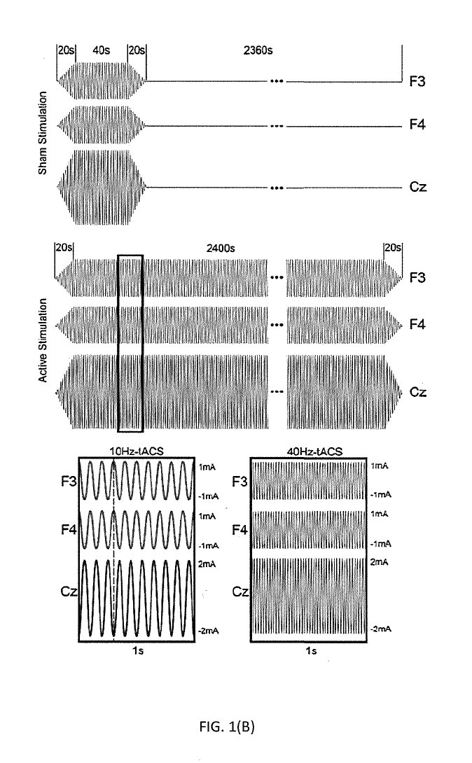

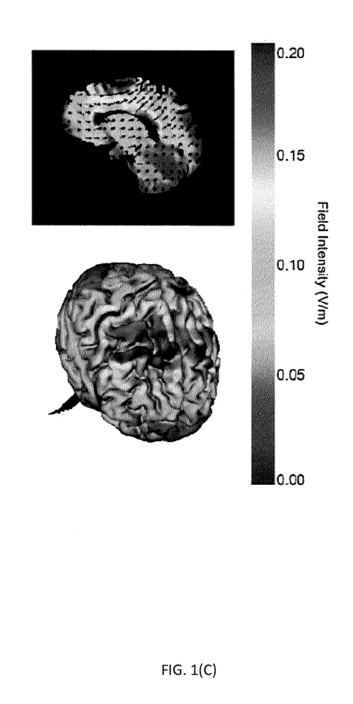

All three study arms used the same electrode montage (FIG. 1). Three electrodes with ten20 paste (Bio-Medical Instruments, Clinton Township, Mich.) were applied to the scalp. Two 5×5 cm electrodes were placed over left and right frontal areas (F3 and F4 respectively in the 10-20 placement system) with a third 5×7 cm “return/reference” electrode placed over the vertex (Cz in the 10-20 system). The electrode montage described here delivers in-phase synchronized stimulation to both the left and right frontal regions to target the imbalance between frontal alpha activity.

Each participant completed 5 consecutive days of the intervention (40 minutes of stimulation) at approximately the same time of day (±90 minutes). The choice of intervention duration (5 consecutive days, 40 minutes each day) was informed by a previous study of transcranial direct current stimulation (tDCS) which found efficacy four weeks after completion of treatment. The tACS stimulation waveform was a sine-wave with an amplitude of 2 mA at Cz and an amplitude of 1 mA at F3 and F4. There were two tACS conditions: the proposed therapeutic frequency of 10 Hz and the control frequency of 40 Hz. Previous research indicates that gamma oscillations have a stronger relationship to cognition and would theoretically not target alpha oscillations and not result in mood symptom changes; therefore, 40 Hz-tACS would be an appropriate control frequency for this trial. Active sham stimulation included 20 seconds of ramp-in to 40 seconds of 10 Hz-tACS, with a ramp-out of 20 seconds, for a total of 80 seconds of stimulation. Both 10 Hz and 40 Hz-tACS also had 20 seconds of ramp-in to 40 minutes of stimulation, with a ramp-out of 20 seconds for a total of 2440 seconds of stimulation (FIG. 1B). During each stimulation session, participants were seated comfortably upright with their eyes open and asked to focus on a ReefScapes video (Undersea Productions, Queensland, Australia) presented on a large projector screen directly in front of them. This video served the purpose of masking the phosphenes induced by tACS and keeping all participants in the same state during stimulation sessions. On the final day of stimulation (Day 5) participants were asked if they believed they received stimulation over the past week (Yes, No, I don't know) to assess blinding.

### High Density Electroencephalography

Resting state EEG (RSEEG) was collected at Day 1 (baseline), Day 5 and the 4 week follow-up, using a 128 channel EEG system (Geodesic EEG system 410, Electrical Geodesics Inc, OR, USA). RSEEG was administered before the final stimulation on Day 5 to avoid recording the immediate aftereffects of tACS. Participants followed pre-programmed computer-generated instructions (Presentation, Neurobehavioral Systems, CA, USA) and had their eyes closed for 2 minutes, following which they had their eyes open for 2 minutes. This sequence was repeated twice resulting in a total of 8 minutes of RSEEG. The sequence was counterbalanced across participants, i.e., half of the participants started with 2 minutes of eyes open condition and the other half started with 2 minutes of eyes closed condition. During the eyes-open condition, participants were instructed to fixate on a cross-hair. Participants also completed a working memory task, the results of which are not presented here.

EEG analysis was performed using EEGLab and custom-written Matlab scripts. Preprocessing consisted of band-pass filtering to 1-50 Hz, downsampling to 250 Hz, removal of bad channels based on low correlations to surrounding channels, and artifact subspace reconstruction followed by independent component analysis to remove artifacts caused by eye blinks, eye movements, muscle activity and heartbeats. EEG data were separated according to the eyes-open and eyes-closed condition and epoched into 10 second segments, following which power spectral density (PSD) was estimated using multi-taper windowed FFT method. To determine changes in RSEEG at Day 5 and the 4 week follow-up, spectral power in the alpha frequency band (8-12 Hz) was calculated and decibel-normalized to spectral power estimated from baseline at each individual electrode as well as averages within a topographical region. The former was used to assess significant changes in oscillation power within each group, while the latter was used to compare changes across the three groups. In addition, alpha power in baseline session was log-transformed and compared between the groups.

### Side Effects and Safety

Side effects were assessed after every stimulation session. Suicidal thoughts/actions were monitored daily with a self-report questionnaire starting from baseline until the 4 week follow-up, as well as during clinical assessments (MADRS and HDRS). Possible development of mania was monitored with the Young Mania Rating Scale (YMRS) administered at every study visit from baseline until the 4 week follow-up. The Montreal Cognitive Assessment (MoCA) was administered at two time points (baseline, the 4 week follow-up) to assess any possible cognitive changes.

### Outcome Measures

The primary outcome measure was defined as the change in depressive symptoms measured by the MADRS from baseline to the 4 week follow-up for the ITT sample. The MADRS was administered prior to stimulation at baseline, after the 5^{th} stimulation on Day 5, at the 2 week follow-up and the four week follow-up. The secondary outcome was the change in raw alpha power measured at the 4 week follow-up relative to baseline for the PP sample. The choice of the 4 week follow-up as the primary outcome was based on a previous trial using electrical stimulation. Exploratory outcome measures were defined as the change in the HDRS and Beck Depression Inventory (BDI). Response to treatment was defined as at least a 50% reduction in symptoms from baseline for each clinical assessment. Remission for the MADRS was defined as scoring ≤9, remission for the HDRS was defined as scoring ≤7, and remission for the BDI was defined as scoring ≤12.

### Statistical Analysis

Custom written scripts in R (R Foundation for Statistical Computing, Vienna, Austria) were used for analysis and are available by request. Libraries used in R included Ime4 (24) and pbkrtest. Differences in demographics and baseline characteristics of the three study arms and the severity of adverse effects were assessed with one-way ANOVA, χ² tests of independence and pairwise t-test with false discovery rate (FDR) correction. Spearman's rank-order correlation was used to assess the possible role of placebo response in the effects observed using a belief of treatments questionnaire, as the data was non-parametric. To assess equality of variance, we used Bartlett's K-squared. We used a linear mixed model analysis with fixed factors of “session” (baseline, the 4 week follow-up) and “condition” (10 Hz-tACS, 40 Hz-tACS, active sham 10 Hz-tACS), with random factor “participant” to account for repeated measures within participants. The choice of a linear mixed model for the main outcome takes into consideration any missing data in our ITT sample. The interaction between “session” and “condition” is defined as the effects of “session” on “condition”. Kenward-Roger approximations were used to calculate p-values and perform F-tests for each factor and their interaction in the mixed model. Effect sizes between groups were calculated using eta-squared (η²), and effect sizes within groups (baseline to Day 5, 2 week follow-up, 4 week follow-up) were calculated using Cohen's d. Differences in rates of response and remission of the three study arms and the success of blinding to treatment arm were assessed with χ² tests of independence.

Statistical analysis of EEG data was performed using custom R scripts and the ‘Imertest’ package which allows fitting linear mixed effects model and uses Satterthwaite's approximation to degrees of freedom to determine the F statistics of the fixed effects. Alpha power change were averaged across electrodes over different topographical regions (frontal, central, occipital, temporal/parietal). Alpha asymmetry was measured as ln

$$( \frac{right alpha power}{lef t alpha power} )$$

of the pooled average of left and right frontal electrodes. Linear mixed effects models were fit with power change as the dependent variable, topographical region and condition as fixed factors and participant as random factor. The residuals of the models were tested for normality using the Shapiro-Wilk test. Two different models were fit for Day 5 and the 4 week follow-up. Post-hoc analysis was performed to get contrasts adjusted using Tukey HSD in the ‘emmeans’ package. Baseline alpha power differences were determined by fitting a linear model with log-transformed baseline alpha power as dependent variable and condition and region as factors. To determine which electrodes exhibited significant power change, we performed a one sample t-test with FDR correction. Spearman correlation coefficient was computed between log transformed baseline alpha scores and changes.

### Sample Size Determination

The target sample size was 30 participants, with n=10 for each arm of the study. This sample was chosen based on funding duration and a focus on feasibility, as this was the first study to use tACS in this population; however, several studies of tACS in healthy populations used a similar sample size to show changes in alpha. A total of 32 participants were randomized (ITT sample), with 26 completing all study sessions (PP sample). Enrollment ended because funding had ended.

### Primary Outcome (MADRS)

In the ITT analysis, MADRS scores for all three groups decreased significantly from baseline to the 4 week follow-up, but there was no significant difference in these changes based upon condition (10 Hz-tACS, 40 Hz-tACS, active sham 10 Hz-tACS) (i.e., there was a significant effect of session (F_{1,28.618}=38.87, p<0.001), but not condition (F_{2,28.681}=0.22, p=0.80) or interaction (F_{2,26.559}=0.65, p=0.53)).

### Secondary Outcome (EEG)

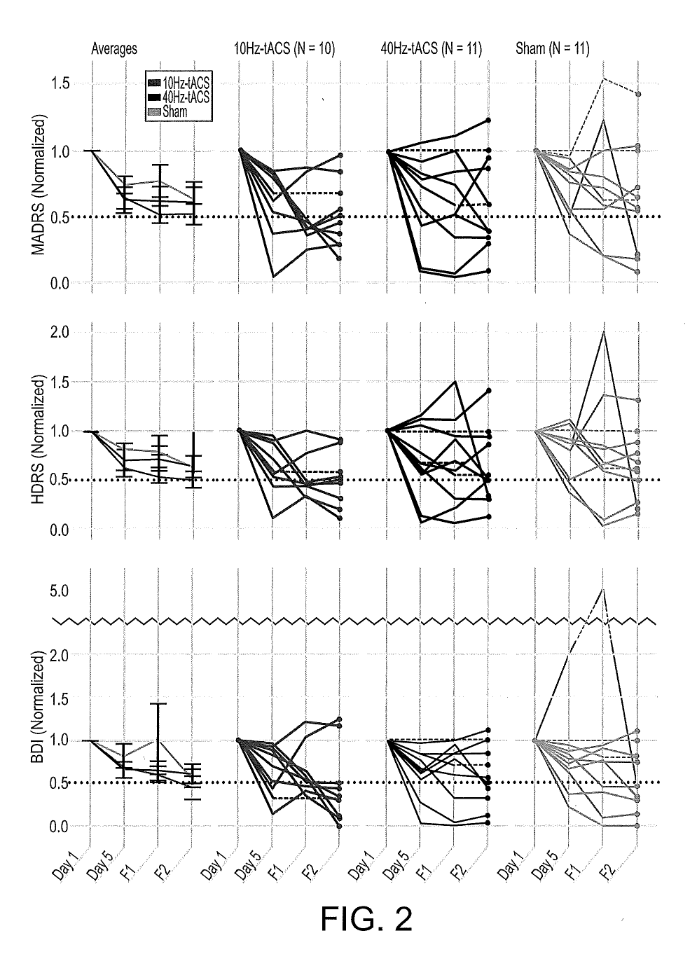

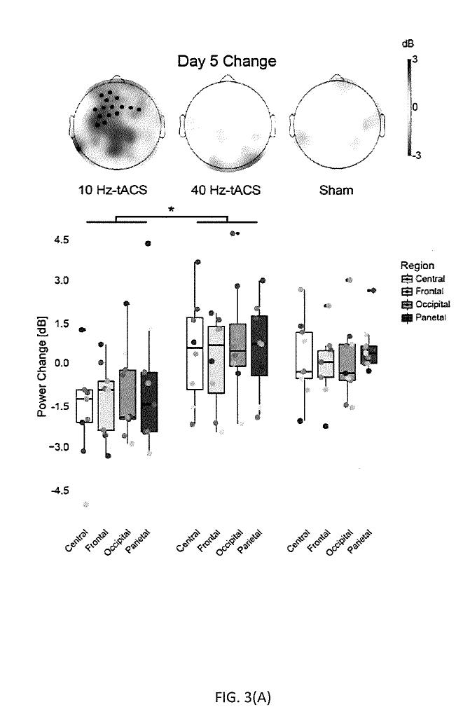

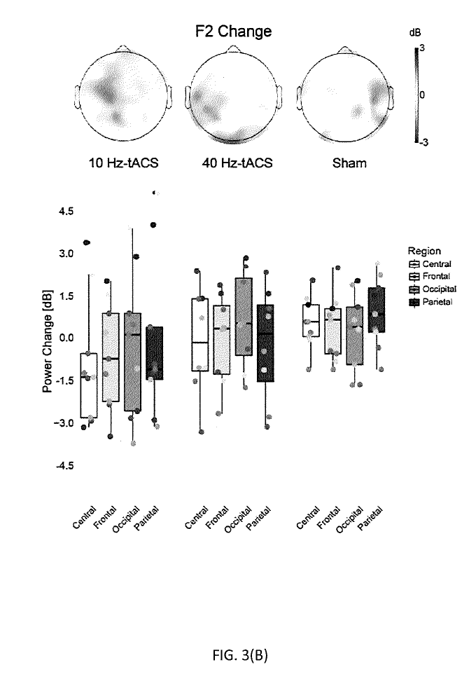

To verify if tACS was effective in engaging alpha oscillations, changes in resting state alpha power at Day 5 (FIG. 3A) and the 4 week follow-up (FIG. 3B) were assessed in the PP sample. Statistical analysis of alpha power change at an individual electrode level as well as region level was performed. Baseline alpha power was not different between the three groups (Linear model factor condition F_{2,92}=0.126, p=0.881; factor region F_{3,92}=3.727, p=0.014, interaction F_{6,92}=0.042, p=0.99), nor was significant alpha asymmetry in our participant sample found (0.0199±0.0336 (mean±sem), p=0.5584 one-sample t-test) or within any treatment groups (10 Hz-tACS: −0.0040±0.0592, p=0.9474; 40 Hz-tACS: 0.0585±0.0548, p=0.3211; sham: 0.0096±0.0639, p=0.8847). Post hoc analysis of contrasts in factor region revealed that the difference between frontal and occipital regions (p=0.020) and the difference between occipital and parietal regions were significant (p=0.039) while the difference between central and occipital regions were trend-level significant (p=0.087). At individual electrode level, the 10 Hz-tACS group showed significant decrease over the left frontal regions (black circles in FIG. 2A, p<0.05, one sample t-test with FDR correction) while no effect of stimulation was observed in the other groups at Day 5 or in any of the groups at the 4 week follow-up. No significant differences were found between the three stimulation conditions on frontal alpha asymmetry on Day 5 relative to Day 1 (F(2,23)=0.24, p=0.7852 one way ANOVA), nor was any significant effect of stimulation on Day 5 relative to Day 1 found within any treatment groups (10 Hz-tACS: 0.0144±0.0330, p=0.6738; 40 Hz-tACS: 0.0145±0.0246, p=0.5739; sham: −0.0171±0.0477, p=0.7289). Significant negative correlations were observed between the log-transformed baseline alpha power and alpha power changes at Day 5 in all the regions (Spearman rank correlations, Frontal ρ=−0.45, p=0.02; Central ρ=−0.54, p<0.01; Parietal ρ=−0.65, p<0.01; Occipital ρ=−0.55, p<0.01). So the log-transformed baseline alpha power was included as a covariate in the analysis of the effects of the intervention. To assess the effect of stimulation on alpha power on Day 5, we fitted a linear mixed model with power change in alpha band as dependent variable and condition (3 levels—10 Hz-tACS, 40 Hz-tACS and Sham) and regions (4 levels—frontal, central, parietal and occipital) as fixed factors and participants as random factors. ANOVA of the model revealed significant effect of condition (F_{2,21,595}=3.931, p=0.035) and region (F_{3,79.358}=7.762, p<0.001). There was no significant effect of interaction (F_{6,68.284}=1.073, p=0.388). Post-hoc analysis of contrasts in factor condition using Tukey HSD revealed significant difference between 10 Hz-tACS group and 40 Hz-tACS group (p=0.039). For factor region, significant differences were found between frontal and occipital (p<0.001), central and occipital (p<0.01), frontal and parietal (p<0.05), while the difference between occipital and parietal was trend-level (p<0.1) ANOVA of the linear mixed model for alpha power changes at the 4 week follow-up revealed significant effect of only region (F_{3,80,094}=2.921, p=0.039). There was no significant effect of condition (F_{2,22.722}=0.629, p=0.542), while interaction was trend-level (F_{6,69.170}=2.019, p=0.075). Post hoc analysis revealed that the differences between frontal and occipital and central and occipital were trend-level (p<0.1). At individual electrode level, the 10 Hz-tACS group showed significant decrease from baseline to Day 5 over the left frontal regions (black circles in FIG. 2A, p<0.05, one sample t-test with FDR correction), while no effect of stimulation was observed in the other groups at Day 5 or in any of the groups at the 4 week follow-up. There was no statistically significant correlation between change in alpha asymmetry and change in MADRS (Spearman's ρ=0.0316, p=0.8784) nor between change in frontal alpha oscillations and change in MADRS (ρ=0.1865; p=0.6397). Taken together, the results indicate that 10 Hz-tACS was effective in targeting alpha oscillations predominantly in the frontal and central regions, but this change did not have a relationship with change in clinical symptoms. Similar analysis of eyes-closed data did not reveal any significant effect of stimulation in alpha power change.

### Response and Remission (MADRS, HDRS, BDI)

Within the ITT sample, a significant relationship was found between condition and response rates for the MADRS (χ²=7.334, df=2, p=0.026) and HDRS (χ²=7.334, df=2, p=0.026) at the 2 week follow-up. This indicated that the 10 Hz-tACS group had a higher rate of response at the 2 week follow-up (77.8%) than the 40 Hz-tACS (30.0%) and sham stimulation groups (20.0%) (FIG. 3). No other significant relationships were found for response rates. Remission rates were also examined over the course of the intervention; however, around half of participants did not qualify for remission at any time point as measured by the MADRS (59% did not remit), the HDRS (50% did not remit), and the BDI (41% did not remit). No significant relationships were found for remission rates.

### Effect Sizes (MADRS, HDRS, BDI)

In exploratory analyses, effect sizes were calculated between and within the treatment groups in the ITT sample. We looked at the mean (SD) change in MADRS from baseline to Day 5, the 2 week follow-up, and the 4 week follow-up. The change in scores from baseline to the 4 week follow-up demonstrated a moderate effect size between conditions (η²=0.060), and the largest effect size was seen in the change from baseline to the 2 week follow-up (η²=0.116). Cohen's d was also calculated in each treatment group from baseline to Day 5, the 2 week follow-up, and the 4 week follow-up. For the MADRS, the largest within group effect size was seen in the 10 Hz-tACS group from baseline to the 2 week follow-up (d=1.70), with the next largest seen in the 10 Hz-tACS group from baseline to the 4 week follow-up (d=1.60). We next sought to validate the effect of tACS on improving MDD symptoms by including analogous rating scales. The HDRS was used as a complementary outcome measure to the MADRS, and the scores were strongly correlated (R²=0.85, p<0.001). The mean (SD) change in HDRS from baseline to the 4 week follow-up was −8.67 (5.32) for 10 Hz-tACS, −5.20 (5.67) for 40 Hz-tACS, and −5.11 (7.61) for sham stimulation. Similar to the MADRS, this change demonstrated a moderate between group effect size (η²=0.071). For the HDRS, the largest within group effect size was seen in the 10 Hz-tACS group from baseline to the 4 week follow-up (d=1.61), with the next largest seen in the 10 Hz-tACS group from baseline to the 2 week follow-up (d=1.58).

Additionally, the BDI was collected as a self-report measure of symptom changes and was strongly correlated with the MADRS(R²=0.72, p<0.001). The mean (SD) change in BDI from baseline to the 4 week follow-up was −14.78 (13.14) for 10 Hz-tACS, −12.20 (11.68) for 40 Hz-tACS, and −12.33 (10.71) for sham stimulation. This change demonstrated a small between-group effect size (η²=0.011). For the BDI, the largest within group effect size was seen in the 10 Hz-tACS group at the 4 week follow-up (d=1.54), with the next largest seen in the sham group from baseline to the 2 week follow-up (d=1.44).

### Safety

One-way ANOVAs were used to determine group differences in the experience and expectations of side effects. Participants from all three groups reported minimal side effects, and there was no difference between the three groups with the exception of “flickering lights” (or phosphenes, p=0.014). Post-hoc paired t-tests with FDR correction were run and found that there was not a significant difference in this side effect between 10 Hz-tACS and 40 Hz-tACS (p=0.99); however, there was a trend-level difference between 10 Hz-tACS and sham (p=0.09) and a significant difference between 40 Hz-tACS and sham (p<0.01). Furthermore, phosphenes were not reported by any of the participants in the sham stimulation group.

After enrollment, 4 participants in the sham stimulation group experienced an increase in suicidal thoughts and one of these 4 participants reported suicidal plans (i.e., scoring greater than 3 on the suicide item on the HDRS). No participants in the 10 Hz-tACS and 40 Hz-tACS groups experienced an increase in suicidal ideation from baseline during the course of the study.

No participants developed mania or hypomania at any point during study participation based on the YMRS. Two adverse events were reported during the course of the study; however, after a thorough investigation, neither were determined to be related to the study or intervention. Finally, participants from all three groups experienced a small improvement in cognition from baseline to the 4 week follow-up as measured by the MoCA. There was no significant difference in this improvement in relation to condition across sessions (F_{2,25.717}=1.354, p=0.276).

### Blinding

Self-report responses from participants on whether they thought they received verum stimulation (Yes, No, I don't know) and the treatment group (10 Hz-tACS, 40 Hz-tACS, sham) were significantly related (χ²=8.304, df=2, p=0.016). Those who received 40 Hz-tACS were more likely to think they had been stimulated, with 90% correctly reporting that they received stimulation. For those who received 10 Hz-tACS, 40% reported that they received stimulation and for those who received sham stimulation, 30% reported that they had received stimulation.

### Expectations of Treatment

One-way ANOVAs were run on expectations of treatment (Item 1: expected likelihood of symptom improvement; Item 2: expected improvement in symptoms) to assess if there were group differences in these expectations of treatment, and there were not (p=0.409, p=0.990, respectively). There was no significant relationship between either of these items and the percent change in MADRS score from baseline to the 2 week follow-up (Item 1: ρ=0.343, p=0.054; Item 2: ρ=0.317, p=0.078) and from baseline to the 4 week follow-up (Item 1: ρ=0.273, p=0.131; Item 2: ρ=0.243, p=0.181). Post-hoc analysis shows that the trend-level relationship between expectations and percent change in MADRS from baseline to the 2 week follow-up was driven by the sham group (Item 1: ρ=0.778, p=0.005; Item 2: ρ=0.693, p=0.018), but not the 10 Hz-tACS (Item 1: ρ=−0.072, p=0.843; Item 2: ρ=0.114, p=0.753) or 40 Hz-tACS groups (Item 1: ρ=0.363, p=0.272; Item 2: ρ=0.080, p=0.816).

This pilot clinical trial was designed to evaluate the feasibility, safety, and preliminary efficacy of alpha tACS as a treatment for the symptoms of MDD. While our primary outcome measure, change in MADRS at the 4 week follow-up in the ITT sample, was not significantly different between the groups, 10 Hz-tACS significantly outperformed 40 Hz-tACS and sham stimulation in terms of response rates at the 2 week follow-up. These results were consistent in both clinician-administered measures (MADRS, HDRS). In addition, 10 Hz-tACS was also effective in engaging alpha oscillations compared to sham or 40 Hz-tACS. Taken together, our results suggest that targeting alpha oscillations with tACS is a potentially viable therapeutic approach and a fully powered subsequent study is justified to further establish tACS as a treatment for depression.

Stimulation was tolerated well by the participants, with no serious adverse events related to the stimulation reported as well as no development of mania, hypomania, or increase in suicidal ideation as a result of stimulation. Retention rates were high for all three groups. These data indicate that future trials using tACS are feasible in this population. The physiological target in this study was frontal alpha oscillations. Resting state recordings in patients with MDD are characterized by increased synchrony in the theta and alpha frequency bands, the latter of which likely emerges from abnormal thalamocortical connectivity. In addition, alpha activity in the left frontal regions has been shown to be higher than that of the right frontal regions. This increased, asymmetric alpha activity is thought to reflect reduced neuronal activity in the left dorsolateral prefrontal cortex, one of several key regions identified as abnormal in brain imaging studies of depression. While repetitive TMS (rTMS) studies for treating depression often use stimulation frequencies around the alpha band, the effect of rTMS on oscillations in patients with MDD has seldom been demonstrated except in a few case studies.

The 10 Hz-tACS led to significantly reduced alpha oscillations over the left frontal regions along with the highest response rates, suggesting that successful reshaping of disrupted oscillations may have led to the decreased symptoms observed, despite the fact that alpha asymmetry was not observed in our sample, indicating that 10 Hz-tACS may have a therapeutic effect regardless of asymmetry. In studies with healthy volunteers, tACS has often been suggested to increase alpha power immediately after stimulation with effects lasting up to 70 minutes. In contrast, the results herein indicate a decrease in alpha power. The differences could be attributed to dosage (5 sessions of 40 minute stimulation vs 1 session of 20 minute stimulation). Alternatively, the differences could be due to the altered network oscillations in patients with MDD (i.e., the impact of tACS may differ in the presence of abnormal alpha activity). While the immediate after-effect of tACS may be enhancement in alpha power, repeated application of tACS may lead to a resetting of oscillators potentially through homeostatic mechanisms resulting in a decrease in alpha power, as is proposed for rTMS. Further studies are required to determine the mechanisms underlying the effect of tACS in patients with aberrant network oscillations.

Despite the promising results as measured by clinician-administered assessments (MADRS, HDRS), the same results in the self-report measure of the BDI were not seen. This may be due to the different time frame (past week vs past 2 weeks) or that patients may have a delay in recognizing symptom changes compared to clinicians. However, the foregoing results indicate tACS may be a feasible and efficacious treatment for MDD.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A method of treating major depressive disorder (MDD) in a patient comprising:
reducing alpha oscillations in one or more frontal brain regions of the patient via administering transcranial alternating current stimulation (tACS) to the patient at a frequency within the alpha frequency band.

2. The method of claim 1, wherein the tACS has a frequency of about 10 Hz.

3. The method of claim 1, wherein the frontal brain regions are targeted by the tACS.

4. The method of claim 3, wherein left and right frontal brain regions are targeted by the tACS.

5. The method of claim 4, wherein the tACS is synchronously administered to the left and right frontal brain regions.

6. The method of claim 1, wherein power of the alpha oscillations is reduced.

7. The method of claim 1, wherein the alpha oscillations exhibit maximum reduction in left frontal brain regions.

8. The method of claim 1, wherein reducing the alpha oscillations reduces alpha oscillation imbalance in left dorsolateral prefrontal cortex.

9. The method of claim 1, wherein alpha oscillations in the left and right frontal brain regions are substantially balanced after the reduction of alpha oscillations in the one or more frontal brain regions.

10. The method of claim 9, wherein power of the alpha oscillations is reduced in the left dorsolateral prefrontal cortex.

11. The method of claim 1 further comprising determining peak alpha oscillation frequency of the patient prior to administration of the tACS.

12. The method of claim 11, wherein the tACS is administered at the peak alpha oscillation frequency of the patient.

13. The method of claim 1, where the reduction in alpha oscillations correlated with a reduction in one or more symptoms of MDD.

14. The method of claim 13, wherein the reduction in alpha oscillations correlated with at least a 50 percent reduction in one or more symptoms of MDD.

15. The method of claim 1, wherein the tACS is administered for a time period of 30 minutes to 60 minutes.

16. The method of claim 1, wherein the tACS is administered via a three electrode system.

17. The method of claim 16, wherein two electrodes at positions F3 and F4 are anodes and a third electrode at position Cz is a cathode.

18. The method of claim 16, wherein the tACS exhibited a sine waveform having amplitude of at least 1 mA.

19. The method of claim 17, wherein the anodes were in-phase and the cathode was opposite phase at any given point in the stimulation.

20. The method of claim 1, wherein the tACS is administered daily for an initial treatment period of at least 5 days.

21. The method of claim 20 further comprising administering the tACS to the patient at one or more intervals after the initial treatment period.

22. The method of claim 9, wherein the alpha oscillations are reduced in one more left frontal brain regions and increased in one or more right frontal brain regions.
